(19) United States
(12) Patent Application Publication
(10) Pub. No.: US 2023/0000729 A1
(43) Pub. Date: Jan. 5, 2023

(54) LIPSTICKS HAVING NATURAL OIL, FATTY ALCOHOL WAX AND FILLER PARTICLES

(71) Applicant: L'OREAL, Paris (FR)

(72) Inventors: Xuefei LIU, Garwood, NJ (US); Trinh NGUYEN, Colonia, NJ (US); Hy Si BUI, Piscataway, NJ (US); Tianyi LIU, Springfield, PA (US)

(73) Assignee: L'OREAL, Paris (FR)

(21) Appl. No.: 17/363,624

(22) Filed: Jun. 30, 2021

(51) Int. Cl. A61K 8/02; A61Q 1/06; A61K 8/92; A61K 8/26

(52) U.S. Cl. CPC A61K 8/0229; A61Q 1/06; A61K 8/922; A61K 8/26; A61K 2800/31; A61K 2800/34; A61K 8/342

(57) ABSTRACT

The invention relates to lipstick compositions including at least one natural oil, at least one fatty alcohol wax and particles of at least one filler, in which the lipstick compositions contain minimal amount of animal-derived ingredients and/or beeswax, or are free of such ingredients, as well as to methods and kits including such corn positions.

### FIELD OF THE INVENTION

The present invention relates to lipstick compositions comprising at least one natural oil, at least one fatty alcohol wax and particles of at least one filler. Preferably, the lipstick compositions contain minimal amount of animal-derived ingredients and/or beeswax, or are free of such ingredients.

### DISCUSSION OF THE BACKGROUND

Many lipstick compositions contain animal derived ingredients or beeswax to achieve a desired texture. Because of growing concerns over how such ingredients may be obtained, many customers prefer to avoid cosmetics containing such ingredients, opting instead for products not relying upon such ingredients. However, without such ingredients (particularly beeswax), it can be difficult to obtain a creamy and desirable texture in a lipstick composition.

U.S. Pat. No. 9,308,397, per the abstract, relates to a cosmetic composition comprising: (A) at least one structured aqueous polymeric adhesive phase comprising: i) at least one aqueous dispersion of adhesive film-forming polymer particles; and ii) at least one structuring agent in an amount effective to viscoelastically thicken, but not solidify, the structured aqueous polymeric adhesive phase; and (B) at least one fatty phase comprising at least one lipidic component wherein (A) is compatible when combined with (B) to form a long-wearing cosmetic composition.

US 2017/0189278 (ES2751661), per the abstract, relates to a cosmetic composition for making up and/or caring for keratin materials, in particular the skin and/or the lips, comprising: at least one aqueous phase gelled with at least one synthetic polymeric hydrophilic gelling agent; and at least one oily phase gelled with at least one lipophilic gelling agent; said phases forming therein a macroscopically homogeneous mixture; said composition also comprising at least one soft-focus filler.

US 2016/0038384, per the abstract, relates to a gel system comprising a fractal network of nanoparticles and macroscopic particles, where the gel system is capable of forming an “optical gel” effective to blurrfine lines and wrinkles as a consequence of the size domain differences between the fractal particles and the macroscopic particles. Cosmetic compositions comprising such gels and methods for their use are disclosed.

U.S. Pat. No. 9,138,388, per the abstract, relates to methods for imparting transfer resistance to a lipstick, comprising: adding to one or more colorants, a coconut alkane mixture comprising a volatile oily composition having from 50 to 100% by weight of a mixture of linear paraffins that include 70 to 99% by weight of at least one linear paraffin selected from C5, C10 and C12 paraffins and mixtures thereof; heating a coconut gel comprising vegetable oil, Cocos Nucifera Oil, a styrene/butadiene copolymer and polyethylene; and adding the colorant and coconut alkane mixture and coconut gel together.

US 2016/0008263 (DE 202015004867), per the abstract, relates to lipstick compositions that are substantive, aesthetically appealing, do not pool on the lips, are easy to spread, and moisturize the lips. The compositions comprise cosmetic ingredients such as conditioning agents, moisturizing agents, antioxidants, structuring agents, emulsifiers, silicone containing compounds, essential oils, thickening agents, preservatives, and colorants, for example.

There remains a need for improved cosmetic compositions having good texture properties, while at the same time minimizing or eliminating the amount of animal-derived ingredients and/or other ingredients with perceived problematic production processes such as beeswax.

Accordingly, one aspect of the present invention is a care and/or makeup and/or treatment composition for lips which has good texture properties while minimizing or eliminating animal-derived ingredients and/or beeswax.

### SUMMARY OF THE INVENTION

The present invention relates to lipstick compositions comprising at least one natural oil, at least one fatty alcohol wax and particles of at least one filler. Preferably, the lipstick compositions contain minimal amount of animal-derived ingredients and/or beeswax, or are free of such ingredients. Preferably, the compositions are anhydrous and in the form of a stick.

The present invention also relates to colored lipstick compositions comprising at least one coloring agent, at least one natural oil, at least one fatty alcohol wax and particles of at least one filler. Preferably, the lipstick compositions contain minimal amount of animal-derived ingredients and/or, or are free of such ingredients. Preferably, the compositions are anhydrous and in the form of a stick.

The present invention also relates to methods of treating, caring for, enhancing the appearance of, and/or making up lips by applying compositions of the present invention to the lips in an amount sufficient to treat, care for, enhance the appearance of, and/or make up the lips.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, an item of clothing or the skin. Transfer resistance may be evaluated by any method known in the art for evaluating such. For example, transfer resistance of a composition may be evaluated by a “kiss” test or material transfer test. This test may involve application of the composition to lips, followed by rubbing a material, for example, a sheet of paper or cotton ball, against the lips after expiration of a certain amount of time following application, such as 2 minutes after application. Similarly, transfer resistance of a composition may be evaluated by the amount of product transferred from a wearer to any other substrate, such as transfer from the lips of an individual to an article of clothing when putting on the clothing after the expiration of a certain amount of time following application of the composition to the lips. The amount of composition transferred to the substrate (e.g., clothing, paper or cotton ball) may then be evaluated and compared. For example, a composition may be transfer resistant if a majority of the product is left on the wearer's lips. Further, the amount transferred may be compared with that transferred by other compositions, such as commercially available compositions. In a preferred embodiment of the present invention, little or no composition is transferred to the substrate from the lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to lips and evaluating the color of the composition after an extended period of time. For example, the color of a composition may be evaluated immediately following application to lips and these characteristics may then be re-evaluated and compared after a certain amount of time such as after 24 hours or 36 hours. Further, these characteristics may be evaluated with respect to other compositions, such as commercially available compositions.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf, including all ranges and subranges therebetween.

This hardness can be measured in one of two ways, for example. A first test for hardness is according to a method of penetrating a probe into the composition and in particular using a texture analyzer (for example, Texture Analyzer model PLUS-UPGRADE from Stable Micro Systems equipped with a stainless steel cylinder of height 28 mm and diameter 2 mm). The hardness measurement is carried out at 20° C. at the center of 5 samples of the composition. The cylinder is introduced into each sample of composition at a pre-speed of 2 mm/s and then at a speed of 0.5 mm/s and finally at a post-speed of 10 mm/s, the total displacement being 2 mm. The recorded hardness value is that of the first maximum peak before the value drops observed.

The second test for hardness is the “cheese wire” method, which involves cutting a 12.4 mm in diameter stick composition and measuring its hardness at 20° C. using a DFGHS 2 tensile testing machine from Indelco-Chatillon Co. or similar machine at a speed of 100 mm/minute. The hardness value from this method is expressed in grams as the shear force required to cut a stick under the above conditions. According to this method, the hardness of compositions according to the present invention which may be in stick form may, for example, range from 120 gf to 270 gf, preferably from 170 gf to 220 gf, for a sample of 12.4 mm in diameter stick.

The hardness of the composition of the present invention may be such that the compositions are self-supporting and can easily disintegrate to form a satisfactory deposit on keratin materials. In addition, this hardness may impart good impact strength to the inventive compositions, which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Low-water-content” means the compositions contain less than 6% water (by weight). Preferably, compositions of the present invention contain less than 5% water, and preferably less than 4% water.

“Anhydrous” means the compositions contain less than 1% water (by weight). Preferably, anydrous compositions of the present invention contain less than 0.5% water, and preferably no water.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of triethanolamine (TEA)” means that TEA is omitted from the composition (that is, 0% by weight), “substantially free of TEA” means that TEA is are present in amounts not greater than 1% by weight, and “devoid of TEA” means that TEA is present in amounts not greater than 0.5% by weight, based on the total weight of the composition. The same nomenclature applies for all other ingredients identified throughout the application such as, for example, silicone oils (“free of volatile silicone oils,” “substantially free of volatile silicone oils,” and “devoid of volatile silicone oils” have meanings consistent with the discussion within this paragraph), even if not specifically discussed for each identified ingredient. Discussed examples of the use of such language are intended to be exemplary, not limiting.

The compositions and methods of the present invention can “comprise,” “consist of” or “consist essentially of” the essential elements and limitations of the invention described herein, as well as any additional or optional ingredients, components, or limitations described herein or otherwise useful. For purposes of the compositions and methods of the present invention where the invention “consists essentially of” the identified ingredients and/or process steps, the “basic and novel property” of such compositions and/or methods is “not having animal derived ingredients and/or beeswax.”

All U.S. patents or patent applications disclosed herein are expressly incorporated by reference in their entirety.

Natural Oil

In accordance with the present invention, lipstick compositions comprising at least one natural oil are provided. For purposes of this application, it should be understood that this definition of “oil” is broader than typical definitions of “oil” and includes substances which are solid at room temperature and at atmospheric pressure but which have melting points as defined herein below 40° C. This is done in part to allow for calculation of weights and ratios disclosed herein and/or to allow for inclusion of higher viscosity materials to be included in the definition of “oil.”

The natural oil(s) are oils recovered or extracted from foods, and/or preferably oils recovered from plants or other vegetative life (as opposed to oils which are derived from natual sources through reactions and which are not “natural oil(s)” as used herein). For example, plant oils include glyceride triesters, which are generally triesters of fatty acids and of glycerol, the fatty acids of which can have varied chain lengths from C4 to C24, it being possible for these chains to be saturated or unsaturated and linear or branched.

Non-limiting examples of suitable natural oil(s) include olive oil, sweet almond oil, coconut oil, avocado oil, wheat germ oil, sunflower oil, grape seed oil, sesame oil, corn oil, apricot oil, castor oil, shea oil, soybean oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, etc., including oils from seeds of the above, and including mixtures thereof. Castor (seed) oil is particularly preferred.

Preferably, the at least one natural oil is/are present in an amount of from about 20% to 60% by weight, preferably from about 25% to about 55% by weight, preferably from about 30% to about 50%, and preferably from about 32% to about 38% by weight relative to the total weight of the composition, including all ranges and subranges therebetween, including for example, 20% to 55%, 30% to 40%, etc.

Fatty Alcohol Wax

In accordance with the present invention, lipstick compositions comprising at least one fatty alcohol wax are provided. The term “wax” means any fatty substance which has at least one melting point (at least one melting peak temperature as determined by DSC (differential scanning calorimetry)) of at least 40° C., and preferably at least 50° C.

Preferably, the fatty alcohol wax contains from about 8 to about 30 carbon atoms, preferably from about 12 to about 28 carbon atoms, and preferably from about 14 to about 24 carbon atoms, including but not limited to 8 to 24 carbon atoms, 12 to 16 carbon atoms, 16 to 22 carbon atoms, etc. The fatty alcohol wax can be saturated or unsaturated, linear or branched.

Mention may be made, for example, of cetyl alcohol, stearyl alcohol, behenyl alcohol, and mixtures thereof. Other examples of suitable fatty alcohol waxes include waxes including natural components (such as from one or more natural oils) and fatty alcohol components such as, for example, hydrogenated stearyl olive ester, commercially available from the supplier Sophim under the tradename Phytowax Olive 18 L 57, and hydrogenated myristyl olive ester, commercially available from the supplier Sophim under the tradename Phytowax Olive 14 L 48.

Preferably, the at least one fatty alcohol wax is/are present in an amount of from about 1% to 15% by weight, preferably from about 1% to about 12% by weight, preferably from about 2% to about 10%, and preferably from about 2% to about 8% by weight relative to the total weight of the composition, including all ranges and subranges therebetween, including for example, 1% to 7%, 2% to 6%, etc.

According to preferred embodiments, more natural oil(s) is/are present in the composition than fatty alcohol wax(es). Accordingly, the natural oil(s) to fatty alcohol wax(es) weight ratio is preferably greater than 1. Preferably, the natural oil(s) to fatty alcohol wax(es) weight ratio is at least 2:1, preferably at least 5:1, preferably at least 6:1 and preferably at least 7:1, including all ranges and subranges therebetween such as, for example, ranges from 2:1 to 30:1, from 10:1 to 25:1, from 2:1 to 8:1, etc.

Filler Particles

In accordance with the present invention, lipstick compositions comprising particles of at least one filler are provided. The filler particles may be spherical, i.e. they may comprise at least a rounded general portion, preferably defining at least a sphere portion, preferably internally defining a concavity or a hollow (sphere, globules, bowls, horseshoe, and the like), or lamellar. Preferably, the filler particles are of natural origin (based on natural substances).

Suitable examples of natural fillers include: cellulose fillers such as, for example, spherical cellulose microparticles, such as Cellulobeads D-10, Cellulobeads D-5 and Cellulobeads USF, sold by the company Daito Kasei Kogyo; and starch fillers such as, for example, crosslinked or non-crosslinked corn, wheat or rice starches such as zea may (corn) starch, cellulose, Oryza sativa (rice) starch, aluminum starch octenylsuccinate, potato starch modified, sodium carboxylmethyl starch.

Another type of natural filler suitable for use in the lipstick compositions of the present invention is/are natural mineral filler particles. Any natural mineral filler particle is suitable for inclusion in the compositions of the present invention. Preferably, the mineral filler particles are of mica or talc, preferably mica, which have not been surfaced-treated, such as, for example, mica sold under the trade name MEARLMICA DD from BASF COLORS AND EFFECTS.

Preferably, the particles of fillers is/are present in an amount of from about 0.25% to 10% by weight, preferably from about 0.5% to about 7% by weight, preferably from about 0.5% to about 5%, and preferably from 1% to 3% by weight relative to the total weight of the composition, including all ranges and subranges therebetween, including for example, 1.5% to 3%, 1.25% to 3.3%, etc.

According to preferred embodiments, more fatty alcohol wax(es) is/are present in the composition than particles of filler(s). Accordingly, the fatty alcohol wax(es) to particles of filler(s) weight ratio is preferably greater than or equal to 1. Preferably, the fatty alcohol wax(es) to particles of filler(s) weight ratio is at least 1:1, preferably at least 1.2:1, preferably at least 1.5:1 and preferably at least 2:1, including all ranges and subranges therebetween such as, for example, ranges from 1:1 to 1.5:1, from 1:1 to 2:1, from 1:1 to 1.75, etc.

According to preferred embodiments, more natural oil(s) is/are present in the composition than particles of filler(s). Accordingly, the natural oil(s) to particles of filler(s) weight ratio is preferably greater than 1. Preferably, the natural oil(s) to particles of filler(s)weight ratio is at least 2:1, preferably at least 5:1, preferably at least 6:1 and preferably at least 10:1, including all ranges and subranges therebetween such as, for example, ranges from 2:1 to 30:1, from 10:1 to 25:1, from 2:1 to 8:1, etc.

Coloring Agents (Colorant)

According to preferred embodiments of the present invention, lipstick compositions further comprising at least one coloring agent are provided.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, surface-treated mica, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agent(s) Is/are present in an amount of from about 1% to 30% by weight, preferably from about 3% to about 25% by weight, preferably from about 5% to about 20%, and preferably from about 8% to about 16% by weight relative to the total weight of the composition, including all ranges and subranges therebetween, including for example, 6% to 16%, 2.5% to 18% 10% to 20%, etc.

If sufficient coloring agent(s) is/are present to provide color to lips after application of the lipstick composition of the present invention to lips, then the composition is preferably used at least as a cosmetic composition for affecting the color appearance of the lips. If insufficient coloring agent(s) is/are present to provide color to lips after application of the lipstick composition of the present invention to lips, then the composition is preferably used at least as a cosmetic composition for aspects other than affecting the appearance of the lips such as providing moisturization or shine or sunscreen protection to the lips (for example, as a non-coloring moisturizing lipstick, a non-coloring topcoat, a non-coloring lipgloss, a non-coloring sunscreen composition, etc.)

Oil Phase

According to embodiments of the present invention, lipstick compositions comprising at least one fatty substance in addition to the at least one natural oil and the at least one fatty alcohol wax are provided. Suitable fatty substances include oil(s) and/or wax(es), with “oil” and “wax” both being defined above.

Suitable additional oils in addition to the natural oils include volatile and/or non-volatile oils. Such oils can be any acceptable oil including but not limited to silicone oils and/or hydrocarbon oils.

Preferably, however, the oil phase of the lipstick composition contains less than 10% by weight of silicone oil(s), preferably less than 5% by weight of silicone oil(s), preferably less than 2.5% by weight of silicone oil(s), or preferably is “substantially free,” “devoid” of or “free” of silicone oil(s) as defined above.

Preferably, however, the oil phase of the lipstick composition contains less than 10% by weight of volatile oil(s), preferably less than 5% by weight of volatile oil(s), preferably less than 2.5% by weight of volatile oil(s), or preferably is “substantially free,” “devoid” of or “free” of volatile oil(s) as defined above.

If present, suitable additional oils in addition to the natural oil(s) include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| cyclopentasiloxane or D5) | | |
| Octamethylcyclotetrasiloxane | 55 | 2.5 |
| cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| 1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₅ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| | Flash Point |
|---|---|
| Compound | (° C.) |
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatility of the solvents/oils can be determined using the evaporation speed as set forth in U.S. Pat. No. 6,338,839, the contents of which are incorporated by reference herein.

According to certain embodiments of the present invention, the composition comprises at least one non-volatile oil. Examples of non-volatile oils that may be used in the present invention include, but are not limited to, polar oils such as, for example:

caprylic/capric acid triglycerides, for instance those sold by the company Stearineries Dubois or those sold under the names Miglyol 810, 812 and 818 by the company Dynamit Nobel;

oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆ +R₇≥10, such as, for example, Purcellin oil (cetostearyl octanoate), isononyl isononanoate, octyldodecyl neopentanoate, C₁₂ to C₁₅ alkyl benzoate, isopropyl myristate, 2-ethylhexyl palm itate, and octanoates, decanoates or ricinoleates of alcohols or of polyalcohols; hydroxylated esters, for instance isostearyl lactate or diisostearyl malate; and pentaerythritol esters;

ethers containing from 10 to 40 carbon atoms;

liquid C₈ to C₂₆ fatty alcohols, for instance oleyl alcohol, cetyl alcohol, stearyl alcohol, octyldodecanol, and cetearly alcohol;

esters having melting points of more than 25° C. and less than 40° C. which function as additional oils, such as bis-diglyceryl polyacyladipate; and

mixtures thereof. Preferred additional oils are esters having melting points of more than 25° C. and less than 40° C., such as bis-diglyceryl polyacyladipate. Although not wishing to be bound by any theory, it is believed that inclusion of such ingredients surprisingly increases hardness of the resulting compositions when combined with the other ingredients of the present invention. Also preferred are liquid C₈ to C₂₆ fatty alcohols such as octyldodecanol.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present invention, the compositions of the present invention comprise at least one silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

According to preferred embodiments, the at least one oil in addition to the natural oil(s) is/are present in the compositions of the present invention in an amount ranging from about 1% to about 45% by weight, more preferably from about 5 to about 42.5% by weight, and preferably from about 10% to about 40% by weight, based on the total weight of the composition, including all ranges and subranges within these ranges such as, for example, 15% to 40%, 20% to 45%, etc.

According to preferred embodiments of the present invention, the compositions of the present invention comprise at least one additional wax in addition to the fatty alcohol wax. Preferably, the at least one additional wax has a melting point of at least 40° C., preferably at least 50° C., and preferably at least 60° C.

Preferably, however, the oil phase of the lipstick composition contains less than 10% by weight of beeswax, preferably less than 5% by weight of beeswax, preferably less than 2.5% by weight of beeswax, or preferably is “substantially free,” “devoid” of or “free” of beeswax as defined above.

Preferably, however, the oil phase of the lipstick composition contains less than 10% by weight of synethetic wax(es), preferably less than 5% by weight of synethetic wax(es), preferably less than 2.5% by weight of synethetic wax(es), or preferably is “substantially free,” “devoid” of or “free” of synethetic wax(es) as defined above.

Preferably, however, the oil phase of the lipstick composition contains less than 10% by weight of silicone wax(es), preferably less than 5% by weight of silicone wax(es), preferably less than 2.5% by weight of silicone wax(es), or preferably is “substantially free,” “devoid” of or “free” of silicone wax(es) as defined above.

Suitable examples of additional waxes that can be used in accordance with the present disclosure include those generally used in the cosmetics field: they include those of natural origin, in particular vegetable waxes, and include waxes such as carnauba wax, candelilla wax, ouricoury wax, Japan wax, sunflower seed wax, cork fibre wax or sugar cane wax, rice wax, montan wax, paraffin wax, lignite wax or microcrystalline wax, ceresin or ozokerite. Sunflower seed wax and candelilla wax are preferred.

Examples of silicone waxes, if present, include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S, alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. patent application 2007/0149703, the entire contents of which is hereby incorporated by reference, with a particular example being C30-C45 alkyldimethylsilyl polypropylsilsesquioxane; and mixtures thereof.

If present, the additional wax or waxes in addition to the fatty alcohol wax may be present in an amount such that the total wax amount (fatty alcohol wax plus additional wax ranges from about 1% to about 30% by weight relative to the total weight of the composition, for example from about 5% to about 25%, and for example from about 10% to about 20%, including all ranges and subranges therebetween as exemplified above with respect to other ingredients, such as, for example, 7.5% to 22.5%, 5% to 17%, etc.

According to preferred embodiments, more additional wax(es) is/are present in the composition than fatty alcohol wax(es). Accordingly, the additional wax(es) to fatty alcohol wax(es) weight ratio is preferably greater than 1. Preferably, the additional wax(es) to fatty alcohol wax(es) weight ratio is at least 2:1, preferably at least 3:1, preferably at least 3.5:1 and preferably at least 4:1, including all ranges and subranges therebetween such as, for example, ranges from 2.5:1 to 5:1, from 3:1 to 6:1, from 2.25:1 to 8:1, etc.

Additional Additives

The composition of the invention can also comprise any additive usually used in the field under consideration. For example, dispersants such as poly(12-hydroxystearic acid), antioxidants, film forming agents, sunscreens, preserving agents, fragrances, fillers, neutralizing agents, cosmetic and dermatological active agents such as, for example, emollients, moisturizers, vitamins, essential fatty acids, surfactants, viscosity increasing agents such as liposoluble/lipodispersible polymers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

According to preferred embodiments of the present invention, methods of treating, enhancing the appearance of, caring for and/or making up lips by applying compositions of the present invention to the lips in an amount sufficient to treat, enhance the appearance of, care for and/or make up the keratinous material are provided. Preferably, “making up” the keratin material includes applying at least one coloring agent to the lips in an amount sufficient to provide color to the lips

In accordance with the preceding preferred embodiments, the compositions of the present invention are applied topically to the lips in an amount sufficient to treat, enhance the appearance of, care for and/or make up the lips. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less.

The present invention also envisages kits and/or prepackaged materials suitable for consumer use containing one or more compositions according to the description herein, alone or in combination with makeup products such as basecoats, topcoats, moisturizing compositions, etc. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1: Sample Composition (Colored Lipstick)

| | AMOUNT (WEIGHT % |
|---|---|
| | OF TOTAL WEIGHT |
| INGREDIENT | OF COMPOSITION) |
| Particles of Natural Fillers | 0.5% to 5% |
| (Cellulose or Mica) | |
| Behenyl Alcohol | 3% to 6% |
| Additional Waxes | 10% to 15% |
| (candelilla and sunflower seed) | |
| Liquid Fatty Alcohol Oil/Ester Oil | 20% to 40% |
| Natural Oils | 30% to 45% |
| Pigments | 8% to 15% |
| Other (e.g., emollients, | 0% to 2% |
| antioxidants, fragrance) | |

### Example 2: Sample Lipstick Composition Preparation

Grind the pigments in the oil phase, until desirable dispersion is achieved.

Heat the pigment grind to 90° C., mix with a propeller mixer.

Add waxes behenyl alcohol, candelilla wax and sunflowerseed wax until melted. Continue to mix for 15 minutes.

Add bis-diglyceryl polyacyladipate after melting (optional).

Add natural filler (cellulose or mica) and mix until homogeneous liquid.

Pour the liquid bulk (at 90° C.) into a room temperature lipstick mold, cool the mold to −18° C. to produce colored lipsticks.

### Example 3: Comparative Testing

Identical compositions were prepared in accordance with the sample composition of example 1 and preparation method of example 2, except for the amount of natural filler particles added as explained below.

A control composition containing no natural filler was prepared. Three compositions containing cellulose particles at 1.5%, 3% and 5% levels were prepared (M1.5, M3 and M5). And three compositions containing mica particles at 1.5%, 3% and 5% levels were also prepared (C1.5, C3 and C5).

Hardness of all compositions prepared was tested using the two testing procedures set forth earlier in the application. Results indicated that the control composition without filler and the compositions having 5% mica or cellulose (M5 or C5) had approximately the same peak positive force (about 280 g). In contrast, compositions containing 1.5% or 3% natural fillers (C1.5, C3 and M3) surprisingly had significantly higher peak positive force (about 290 g or above), indicating compositions with better hardness properties. M1.5 (1.5% mica), however, had poorer hardness properties than even the control composition.

What is claimed is:

1. A lipstick composition comprising at least one natural oil, at least one fatty alcohol wax and particles of at least one natural filler.

2. The composition of claim 1, wherein the at least one fatty alcohol wax has a melting point of at least 50° C.

3. The composition of claim 1, further comprising at least one additional wax in addition to the at least one fatty alcohol wax.

4. The composition of claim 3, wherein the weight ratio of additional wax(es) to fatty alcohol wax(es) is at least 4:1, the fatty alcohol wax(es) to particles of filler(s) weight ratio is at least 1:1, and the weight ratio of the natural oil(s) to fatty alcohol wax(es) is at least 2:1.

5. The composition of claim 1, further comprising at least one coloring agent.

6. The composition of claim 1, further comprising at least one additional oil in addition to the at least one natural oil, wherein the additional oil is at least one liquid C₈ to C₂₆ fatty alcohol, preferably octyldodecanol.

7. The composition of claim 1, in the form of a stick.

8. The composition of claim 1, wherein the composition is anhydrous.

9. The composition of claim 1, wherein the composition is free of beeswax.

10. The composition of claim 1, wherein the composition satisfies one or more of the following: (a) the composition is free of volatile oil; (b) the composition is free of silicone oil; (c) the composition is free of silicone wax.

11. The composition of claim 1, wherein the composition satisfies all of the following: (a) the composition is free of volatile oil; (b) the composition is free of silicone oil; (c) the composition is free of silicone wax.

12. The composition of claim 1, wherein the fatty alcohol wax is behenyl alcohol and the natural filler is selected from the group consisting of cellulose, mica which has not been surface-treated, and mixtures thereof.

13. The composition of claim 1, wherein the natural filler is selected from the group consisting of cellulose, mica which has not been surface-treated, and mixtures thereof, and the natural filler is present in an amount of about 0.5% to about 5% by weight with respect to the total weight of the composition.

14. A method of caring for or making up lips comprising applying the lipstick composition of claim 1 to the lips.

15. A method of making up lips comprising applying the lipstick composition of claim 5 to the lips.
